(19) United States
(12) Patent Application Publication
(10) Pub. No.: US 2021/0030062 A1
(43) Pub. Date: Feb. 4, 2021

(54) Aerosol Generating Article, An Aerosol Generating System And A Method For Generating A Flavoured Aerosol

(71) Applicant: JT International S.A., Geneva (CH)

(72) Inventor: Andrew Robert John Rogan, Forres (GB)

(73) Assignee: JT International S.A., Geneva (CH)

(21) Appl. No.: 17/043,880

(22) PCT Filed: May 28, 2019
(86) PCT No.: PCT/EP2019/063787

§ 371 Date: Sep. 30, 2020

(30) Foreign Application Priority Data
May 31, 2018 (EP) 18175298.1

(51) Int. Cl. A24F 40/30; A24F 40/10; A24F 40/20; A24F 40/44; A24F 40/42; A24F 40/465; H05B 6/10

(52) U.S. Cl. CPC A24F 40/30; A24F 40/10; A24F 40/20; H05B 6/106; A24F 40/42; A24F 40/465; H05B 6/108; A24F 40/44

(57) ABSTRACT

An aerosol generating article comprises includes a reservoir for an aerosol forming liquid, a liquid absorbing material for absorbing aerosol forming liquid from the reservoir and a non-liquid flavour generating material positioned outside the reservoir The liquid absorbing material and the non-liquid flavour generating material are arranged to be heated simultaneously when the aerosol generating article is positioned in an aerosol generating device to generate a flavoured aerosol for inhalation by a user. An aerosol generating system and a method for generating a flavoured aerosol are also described.

### TECHNICAL FIELD

The present disclosure relates generally to an aerosol generating article, and more particularly to an aerosol generating article for use with an aerosol generating device for heating the aerosol generating article to generate an aerosol for inhalation by a user. Embodiments of the present disclosure also relate to an aerosol generating system and to a method for generating a flavoured aerosol.

### TECHNICAL BACKGROUND

Aerosol generating systems (also known as electronic cigarettes, e-cigarettes, personal vaporisers and electronic vapour inhalers), which can be used as an alternative to conventional smoking articles such as lit-end cigarettes, cigars, and pipes, have become popular with consumers in recent years. Various approaches to aerosol generation can be employed in such systems.

In one approach, a flavoured aerosol forming liquid is heated to produce a flavoured aerosol which can be inhaled by a user. In another approach, a non-liquid flavour generating material, such as tobacco, containing an aerosol former is heated to generate a flavoured aerosol which can be inhaled by a user. With both of these approaches, the aerosol is typically inhaled through a mouthpiece to deliver the aerosol to the lungs.

Both of these approaches to aerosol generation have drawbacks which the present disclosure seeks to mitigate.

### SUMMARY OF THE DISCLOSURE

According to a first aspect of the present disclosure, there is provided an aerosol generating article comprising:
a reservoir for an aerosol forming liquid;
a liquid absorbing material for absorbing aerosol forming liquid from the reservoir; and
a non-liquid flavour generating material positioned outside the reservoir;
wherein the liquid absorbing material and the non-liquid flavour generating material are arranged to be heated simultaneously when the aerosol generating article is positioned in an aerosol generating device.

The aerosol generating article is for use with an aerosol generating device for heating the liquid absorbing material and the non-liquid flavour generating material without burning the non-liquid flavour generating material. The liquid absorbing material and the non-liquid flavour generating material are distinct from each other. Heating of the liquid absorbing material heats and atomises the aerosol forming liquid absorbed by the liquid absorbing material whilst the simultaneous heating of the non-liquid flavour generating material releases volatile compounds including, for example, nicotine or flavour compounds such as tobacco flavouring. An aerosol suitable for inhalation by a user of the aerosol generating device is, thus, generated by the simultaneous heating of the liquid absorbing material and the non-liquid flavour generating material.

In general terms, a vapour is a substance in the gas phase at a temperature lower than its critical temperature, which means that the vapour can be condensed to a liquid by increasing its pressure without reducing the temperature, whereas an aerosol is a suspension of fine solid particles or liquid droplets, in air or another gas. It should, however, be noted that the terms ‘aerosol’ and ‘vapour’ may be used interchangeably in this specification, particularly with regard to the form of the inhalable medium that is generated for inhalation by a user.

Only a proportion of the total amount of aerosol forming liquid in the reservoir is heated and atomised during use of the aerosol generating article in an aerosol generating device, namely the aerosol forming liquid absorbed by the liquid absorbing material which is typically sufficient for a single inhalation by a user of the aerosol generating device. Thus, the amount of energy required to atomise the aerosol forming liquid and generate an aerosol for inhalation by a user can be minimised because only an amount of aerosol forming liquid that is required for a single inhalation is heated and atomised. This is to be contrasted with the conventional approach mentioned above in which a non-liquid flavour generating material comprises an aerosol-former in an amount required for multiple inhalations that would normally take place during a smoking session and, therefore, which requires a much larger energy input to generate an aerosol.

The aerosol forming liquid may comprise polyhydric alcohols and mixtures thereof such as glycerine or propylene glycol.

The liquid absorbing material may comprise a porous ceramic, a fibre bundle, a capillary tube or a wicking material. The liquid absorbing material may comprise a porous ceramic wick. The liquid absorbing material may contact the aerosol forming liquid in the reservoir to enable absorption of the aerosol forming liquid by the liquid absorbing material, for example due to capillary action or wicking.

The non-liquid flavour generating material may be any type of solid or semi-solid material. Example types of solid or semi-solid materials include granules, pellets, powder, shreds, strands, particles, gel, strips, loose leaves, cut filler, porous material, foam material or sheets. The non-liquid flavour generating material may comprise plant derived material and in particular, may comprise tobacco.

The aerosol generating article may comprise an inductively heatable susceptor. The liquid absorbing material and the non-liquid flavour generating material may be arranged to be heated simultaneously by the inductively heatable susceptor when the aerosol generating article is positioned in an aerosol generating device. The use of an inductively heatable susceptor provides a convenient, effective and energy efficient way to heat the liquid absorbing material and the non-liquid flavour generating material. When the aerosol generating article is positioned in an aerosol generating device and exposed to a time varying electromagnetic field, heat is generated in the inductively heatable susceptor due to eddy currents and magnetic hysteresis losses resulting in a conversion of energy from electromagnetic to heat. The heat generated in the inductively heatable susceptor is transferred to the liquid absorbing material and the non-liquid flavour generating material, ensuring that they are simultaneously heated to generate an aerosol with the desired characteristics.

The inductively heatable susceptor may comprise one or more, but not limited, of aluminium, iron, nickel, stainless steel and alloys thereof, e.g. Nickel Chromium or Nickel Copper. The inductively heatable susceptor may comprise a particulate susceptor material.

The inductively heatable susceptor may be distributed in the liquid absorbing material and may be distributed substantially evenly in the liquid absorbing material. Uniform heating of the liquid absorbing material is thereby achieved. Substantially even distribution of the inductively heatable susceptor in the liquid absorbing material may also allow the aerosol generating article to be easily manufactured.

The inductively heatable susceptor may be distributed in the non-liquid flavour generating material and may be distributed substantially evenly in the non-liquid flavour generating material. Uniform heating of the non-liquid flavour generating material is thereby achieved. Substantially even distribution of the inductively heatable susceptor in the non-liquid flavour generating material may also allow the aerosol generating article to be easily manufactured.

In some embodiments, the non-liquid flavour generating material may be distributed in the liquid absorbing material and may be distributed substantially evenly in the liquid absorbing material. Simultaneous and uniform heating of both the liquid absorbing material and the non-liquid flavour generating material are achieved, thereby resulting in the generation of an aerosol with uniform and repeatable characteristics, such as flavour.

In embodiments in which the non-liquid flavour generating material is distributed in the liquid absorbing material, the aerosol generating article may be manufactured by sintering the liquid absorbing material at low temperature. More particularly, the non-liquid flavour generating material may be mixed with the liquid absorbing material or precursor(s) thereto prior to sintering the mixture at low temperature. Sintering at low temperature advantageously ensures that during manufacture of the aerosol generating article, the non-liquid flavour generating material is not heated to a temperature at which it releases volatile compounds. Suitable manufacturing methods based on low-temperature sintering, and in particular where the liquid absorbing material is a porous ceramic, are described in WO 2017/149288 A1, U.S. Pat. No. 9,648,909 B2 and US 2015/359262 A1.

In other embodiments, the non-liquid flavour generating material and the liquid absorbing material may be arranged separately. Optionally, the liquid absorbing material may be arranged to be positioned closer to an atomiser, for example a heater (e.g. a resistive heater) or an induction coil, than the non-liquid flavour generating material when the aerosol generating article is positioned in an aerosol generating device. With this arrangement, the liquid absorbing material may be heated to a higher temperature than the non-liquid flavour generating material, thereby ensuring that an aerosol with optimum characteristics, such as flavour, is generated during use of the aerosol generating article in an aerosol generating device and ensuring that the non-liquid flavour generating material is heated without being burned.

The non-liquid flavour generating material may include an aerosol forming liquid in an amount less than approximately 20% on a dry weight basis, optionally in an amount less than approximately 13% on a dry weight basis, optionally in an amount less than approximately 8% on a dry weight basis. The non-liquid flavour generating material may be impregnated with the aerosol forming liquid. This ensures that the non-liquid flavour generating material retains its solid or semi-solid form. The aerosol forming liquid acts as an aerosol-former and may help to ensure that an aerosol with optimum characteristics is generated during use of the aerosol generating article in an aerosol generating device. The low content of the aerosol forming liquid helps to minimise the amount of energy required for aerosol generation because the majority of the total aerosol content of the aerosol generated during use of the aerosol generating article, for example greater than approximately 85% of the total aerosol content of the aerosol generated during a single inhalation, is generated by heating and atomising the aerosol forming liquid absorbed by the liquid absorbing material.

The non-liquid flavour generating material may include water in an amount less than approximately 15% on a dry weight basis, optionally in an amount less than 8% on a dry weight basis. The non-liquid flavour generating material may be impregnated with the water. This ensures that the non-liquid flavour generating material retains its solid or semi-solid form. The inclusion of water in the non-liquid flavour generating material may ensure that an aerosol with optimum characteristics, and in particular flavour, is generated during use of the aerosol generating article in an aerosol generating device. It may also help to provide the non-liquid flavour generating material with a favourable physical character that facilitates handling and manufacture.

The non-liquid flavour generating material may not be positioned downstream of the liquid absorbing material relative to an aerosol flow direction within the article. For example, the non-liquid flavour generating material may be positioned upstream of and/or alongside the liquid absorbing material. Thus, the aerosol generated by heating the liquid absorbing material, and in particular by heating and atomising the aerosol forming liquid absorbed by the liquid absorbing material, is mixed with one or more volatile components, such as flavour compounds, released during simultaneous heating of the non-liquid flavour generating material. This may additionally prevent or mitigate degradation of the non-liquid flavour generating material by the aerosol generated by heating the liquid absorbing material, in particular because the flow of aerosol through the non-liquid flavour generating material is avoided or at least minimised.

According to a second aspect of the present disclosure, there is provided an aerosol generating system comprising:
an aerosol generating device comprising a cavity; and
an aerosol generating article as defined above positioned in the cavity;
the aerosol generating device further comprising an atomiser for heating the aerosol generating article to generate an aerosol from aerosol forming liquid absorbed in the liquid absorbing material from the reservoir and to generate a flavour from the non-liquid flavour generating material.

As explained above, the energy consumption of the aerosol generating system, and in particular of the atomiser, is minimised when compared to conventional approaches because an aerosol with optimum characteristics is generated by heating and atomising only the aerosol forming liquid absorbed by the liquid absorbing material and by simultaneously heating the non-liquid flavour generating material.

The atomiser may comprise a resistive heater, which may comprise a resistive heating element. The atomiser may comprise an electrode which may be arranged to supply power to a resistive heater which may, for example, form part of the aerosol generating article. The atomiser may comprise a nebuliser.

The atomiser may comprise an induction coil. The induction coil may be arranged to generate an alternating time varying electromagnetic field for inductively heating the inductively heatable susceptor.

The induction coil may comprise a Litz wire or a Litz cable. It will, however, be understood that other materials could be used. The induction coil may be substantially helical in shape and may extend around the cavity.

The circular cross-section of a helical induction coil may facilitate the insertion of the aerosol generating article into the cavity of the aerosol generating device and may promote uniform heating. The resulting shape of the device is also comfortable for the user to hold.

According to a third aspect of the present disclosure, there is provided a method for generating a flavoured aerosol, the method comprising:
providing an aerosol generating article comprising a reservoir for an aerosol forming liquid, a liquid absorbing material and a non-liquid flavour generating material positioned outside the reservoir;
transferring aerosol forming liquid from the reservoir to the liquid absorbing material;
generating an aerosol from the aerosol forming liquid absorbed in the liquid absorbing material;
generating a flavour from the non-liquid flavour generating material; and
mixing the aerosol and the flavour to generate a flavoured aerosol.

The method provides a particularly effective way to generate an aerosol whilst minimising energy consumption as explained above.

The step of generating an aerosol from the aerosol forming liquid absorbed in the liquid absorbing material may comprise heating the liquid absorbed in the liquid absorbing material. The energy required for aerosol generation is thereby minimised when compared to the conventional approaches mentioned above.

The step of generating a flavour from the non-liquid flavour generating material may comprise heating the non-liquid flavour generating material. Heating of the non-liquid flavour generating material directly releases volatile compounds, including flavour compounds, which are mixed with the aerosol generated by heating the liquid absorbed in the liquid absorbing material. A flavoured aerosol with optimum characteristics is thereby generated.

The steps of heating the liquid absorbed in the liquid absorbing material and heating the non-liquid flavour generating material may be performed simultaneously. This further helps to minimise energy consumption.

The step of generating an aerosol from the aerosol forming liquid absorbed in the liquid absorbing material may provide greater than approximately 85% of the total aerosol content of the flavoured aerosol. The step of generating an aerosol from the aerosol forming liquid absorbed in the liquid absorbing material may provide greater than approximately 90% of the total aerosol content of the flavoured aerosol. The step of generating an aerosol from the aerosol forming liquid absorbed in the liquid absorbing material may provide greater than approximately 95% of the total aerosol content of the flavoured aerosol. Thus, it will be understood that the majority of the total aerosol content is generated by heating the aerosol forming liquid absorbed in the liquid absorbing material. The remainder of the aerosol content may, for example, be generated by the step of generating a flavour from the non-liquid flavour generating material, for example by heating the non-liquid flavour generating material. This ensures that the amount of energy required for aerosol generation is minimised.

The aerosol generated by the step of generating an aerosol from the aerosol forming liquid absorbed in the liquid absorbing material typically does not flow through the non-liquid flavour generating material. Thus, the aerosol is not employed to indirectly release volatile compounds from the non-liquid flavour generating material. Instead, volatile compounds, including flavour compounds, are released directly from the non-liquid flavour generating material, for example by heating the non-liquid flavour generating material. This may prevent or mitigate degradation of the non-liquid flavour generating material by the aerosol generated by heating the liquid absorbing material.

### BRIEF DESCRIPTION OF THE DRAWINGS

FIG. 1 is a diagrammatic view of a first embodiment of an aerosol generating system;

FIG. 2 is a diagrammatic view of a second embodiment of an aerosol generating system;

FIG. 3 is a diagrammatic view of a third embodiment of an aerosol generating system; and

FIG. 4 is a diagrammatic view of a fourth embodiment of an aerosol generating system.

### DETAILED DESCRIPTION OF EMBODIMENTS

Embodiments of the present disclosure will now be described by way of example only and with reference to the accompanying drawings.

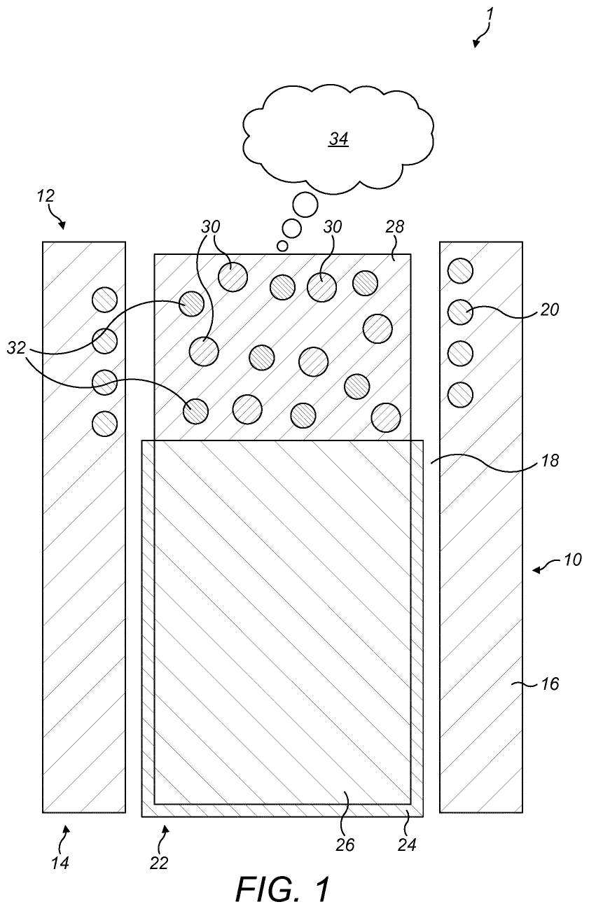

Referring initially to FIG. 1, there is shown diagrammatically a first embodiment of an aerosol generating system 1. The aerosol generating system 1 comprises an aerosol generating device 10 and an aerosol generating article 22. The aerosol generating device 10 has a proximal end 14 and a distal end 12 and comprises a device body 16 which includes a power source and a controller (not shown) which may be configured to operate at high frequency. The power source typically comprises one or more batteries which could, for example, be inductively rechargeable.

The aerosol generating device 10 is generally cylindrical and comprises a generally cylindrical cavity 18 in the device body 16 which extends between the proximal and distal ends 14, 12 of the aerosol generating device 10. The cavity 18 is arranged to receive a correspondingly shaped generally cylindrical aerosol generating article 22 which will be described in further detail below.

The aerosol generating device 10 comprises a helical induction coil 20 which has a circular cross-section and which is positioned in the device body 16 to extend around the cavity 18. The induction coil 20 can be energised by the power source and controller. The controller includes, amongst other electronic components, an inverter which is arranged to convert a direct current from the power source into an alternating high-frequency current for the induction coil 20.

The generally cylindrical aerosol generating article 22 comprises a reservoir 24 for storing an aerosol forming liquid 26, such as glycerine or propylene glycol. The aerosol generating article 22 further comprises a liquid absorbing material 28, such as a porous ceramic, which contacts the aerosol forming liquid 26 in the reservoir 24 so that the aerosol forming liquid 26 in the reservoir 24 is absorbed by the liquid absorbing material 28, for example due to wicking.

The aerosol generating article 22 also comprises a non-liquid flavour generating material 30 which in the illustrated first embodiment is distributed within the liquid absorbing material 28. The non-liquid flavour generating material 30 may be in the form of granules, particles, gel, strips, loose leaves, cut filler, pellets, powder, shreds, strands, foam material or sheets. The non-liquid flavour generating material 30 may comprise tobacco. The non-liquid flavour generating material 30 is advantageously impregnated with an aerosol forming liquid and/or water so that it has some moisture content and is not a completely dry material.

The aerosol generating article 22 comprises an inductively heatable susceptor 32 in particulate form which is also distributed within the liquid absorbing material 28. When a time varying electromagnetic field is produced in the vicinity of the particles of susceptor 32 by the induction coil 20, heat is generated in the susceptor 32 due to eddy currents and magnetic hysteresis losses. The heat is transferred from the particles of susceptor 32 to the liquid absorbing material 28 and the aerosol forming liquid 26 absorbed by the liquid absorbing material 28 is heated to atomise it and thereby generate an aerosol. At the same time, the heat is transferred from the particles of heated susceptor 32 to the non-liquid flavour generating material 30. The non-liquid flavour generating material 30 is thereby heated without being burned and releases volatile compounds, for example including nicotine or flavour compounds such as tobacco flavouring.

The aerosol generated by heating the aerosol forming liquid 26 absorbed by the liquid absorbing material 28 and the flavour compounds generated by simultaneously heating the non-liquid flavour generating material 30 are combined and form a flavoured aerosol 34 which is inhaled by a user of the device 10, for example through a mouthpiece (not shown) fitted to the proximal end 14 of the device 10. It will be understood by one of ordinary skill in the art that the device 10 includes one or more air flow passages that deliver the flavoured aerosol from the distal end 12 to the mouthpiece fitted to the proximal end 14.

When the aerosol forming liquid 26 is atomised due to heat transfer from the heated particles of susceptor 32, it will be understood that further aerosol forming liquid 26 is absorbed by the liquid absorbing material 28 from the reservoir 24, for example due to wicking, so that the absorbed aerosol forming liquid 26 can again be heated in the manner described above at the same time as the non-liquid flavour generating material 30 is heated to generate a flavoured aerosol 34 for inhalation by a user. With this approach, it will be understood that only a small proportion of the aerosol forming liquid 26 within the aerosol generating article 22, namely the aerosol forming liquid 26 absorbed by the liquid absorbing material 28, is heated to generate the aerosol required for a single user-inhalation or puff, as opposed to the whole contents of the reservoir 24, thus minimising the energy required for aerosol generation. Due to the positioning of the mouthpiece at the proximal end 14, it will be understood that during typical use of the device 10 the distal end 12 will be positioned lower than the proximal end 14, thus ensuring that the aerosol forming liquid 26 in the reservoir 24 flows towards, and remains in contact with, the liquid absorbing material 28 as the aerosol forming liquid is depleted.

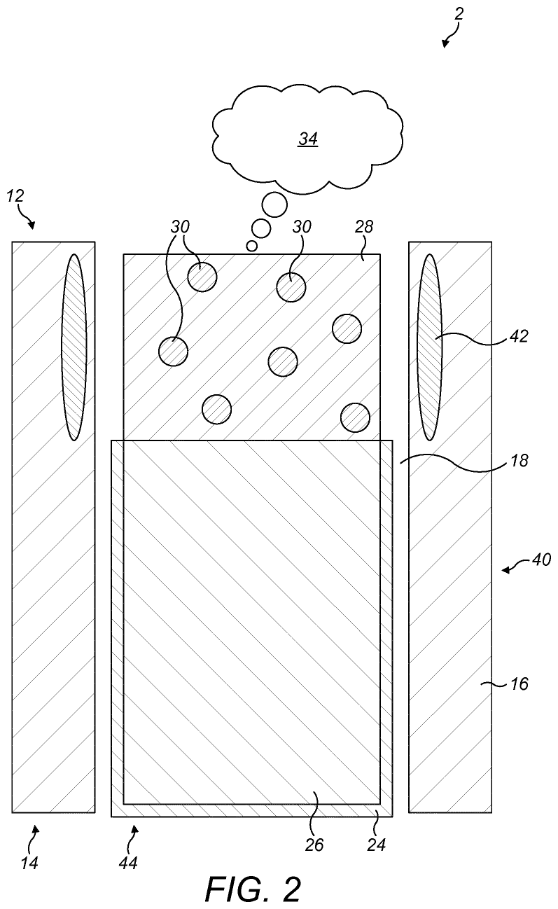

Referring now to FIG. 2, there is shown diagrammatically a second embodiment of an aerosol generating system 2 which is similar to the aerosol generating system 1 described above with reference to FIG. 1 and in which corresponding elements are designated using the same reference numerals.

Unlike the aerosol generating system 1, the aerosol generating system 2 does not use induction heating to heat the aerosol forming liquid 26 absorbed by the liquid absorbing material 28 or to heat the non-liquid flavour generating material 30. Instead, the aerosol generating system 2 comprises an aerosol generating device 40 which includes a resistive heater 42, for example comprising a resistive heating element, positioned in the device body 16 to extend around the cavity 18.

The aerosol generating system further comprises an aerosol generating article 44 which is positioned in the cavity 18. The aerosol generating article 44 is similar to the aerosol generating article 22 described above with reference to FIG. 1, except that it does not include an inductively heatable susceptor 32. An inductively heatable susceptor 32 is not needed due to the resistive heating that is employed by the aerosol generating system 2.

When an electric current is supplied to the resistive heater 42, it heats up and the heat is transferred to the liquid absorbing material 28, for example by radiation and convection, to heat it. Upon heating of the liquid absorbing material 28, the aerosol forming liquid 26 absorbed by the liquid absorbing material 28 is heated to atomise it and thereby generate an aerosol in the manner described above. Similarly, the non-liquid flavour generating material 30 distributed within the liquid absorbing material 28 is simultaneously heated without being burned by the heat transferred from the resistive heater 42, thus causing it to release volatile compounds, for example including nicotine or flavour compounds such as tobacco flavouring.

As explained above, the aerosol generated by heating the aerosol forming liquid 26 absorbed by the liquid absorbing material 28 and the flavour compounds generated by simultaneously heating the non-liquid flavour generating material 30 are combined and form a flavoured aerosol 34 which is inhaled by a user of the device 40, for example through a mouthpiece (not shown) fitted to the proximal end 14 of the device 40.

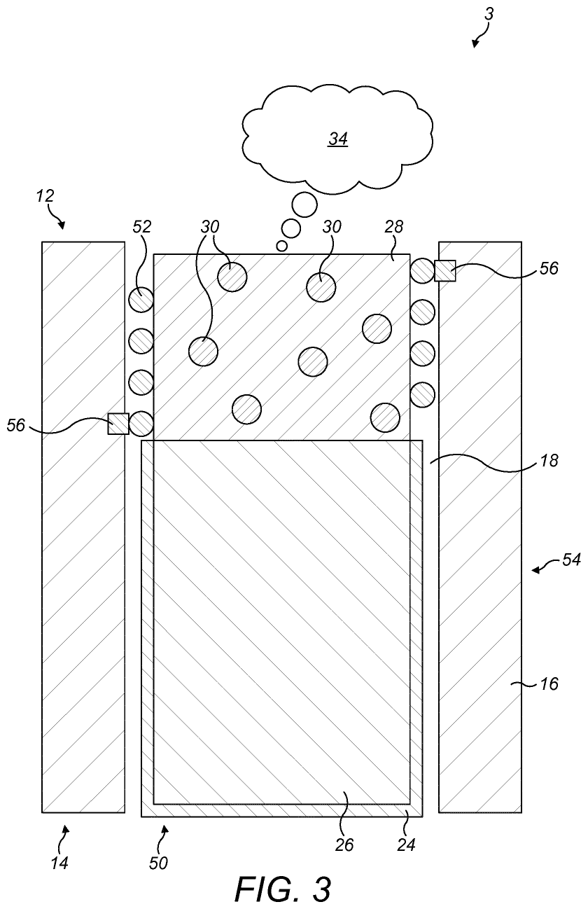

Referring now to FIG. 3, there is shown diagrammatically a third embodiment of an aerosol generating system 3 which is similar to the aerosol generating system 2 described above with reference to FIG. 2 and in which corresponding elements are designated using the same reference numerals.

Like the aerosol generating system 2, the aerosol generating system 3 operates based on the principle of resistive heating. In this embodiment, however, the aerosol generating article 50 comprises a resistive heater 52, for example in the form of a helical resistance heating element, which extends around and contacts the liquid absorbing material 28 which may comprise a bundle of glass fibres. Furthermore, the aerosol generating system 3 comprises an aerosol generating device 54 having electrodes 56 in the device body 16 which are arranged to make electrical contact with the resistive heater 52 when the aerosol generating article 50 is positioned in the cavity 18 in the device body 16.

In operation, an electric current is supplied to the resistive heater 52 via the electrodes 56, causing it to heat up. The heat from the resistive heater 52 is transferred to the liquid absorbing material 28, primarily by conduction but also by radiation and convection, to thereby heat the liquid absorbing material 28. Upon heating of the liquid absorbing material 28, the aerosol forming liquid 26 absorbed by the liquid absorbing material 28 is heated to atomise it and thereby generate an aerosol in the manner described above.

Similarly, the non-liquid flavour generating material 30 distributed within the liquid absorbing material 28 is simultaneously heated without being burned by the heat transferred from the resistive heater 52, thus causing it to release volatile compounds, for example including nicotine or flavour compounds such as tobacco flavouring.

As explained above, the aerosol generated by heating the aerosol forming liquid 26 absorbed by the liquid absorbing material 28 and the flavour compounds generated by simultaneously heating the non-liquid flavour generating material 30 are combined and form a flavoured aerosol 34 which is inhaled by a user of the device 54, for example through a mouthpiece (not shown) fitted to the proximal end 14 of the device 54.

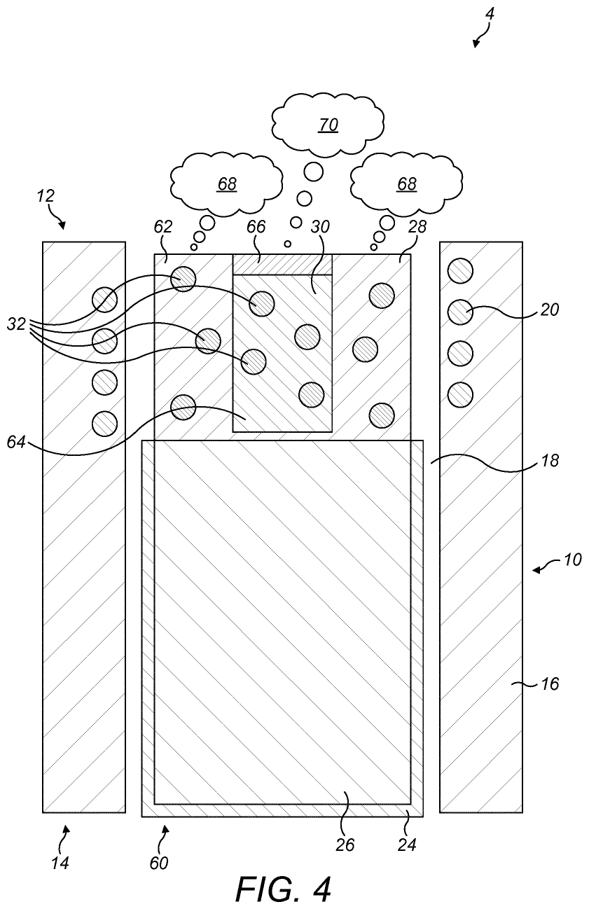

Referring now to FIG. 4, there is shown diagrammatically a fourth embodiment of an aerosol generating system 4 which is similar to the aerosol generating system 1 described above with reference to FIG. 1 and in which corresponding elements are designated using the same reference numerals.

In the aerosol generating system 4, the aerosol generating device 10 is as described above with reference to FIG. 1 and operates based on the induction heating principle. The aerosol generating system 4 comprises an aerosol generating article 60 whose construction differs from the aerosol generating article 22 described above with reference to FIG. 1, as will now be described.

The aerosol generating article 60 comprises a liquid absorbing material 28 which contacts the aerosol forming liquid 26 in the reservoir 24 so that the aerosol forming liquid 26 in the reservoir 24 can be absorbed by the liquid absorbing material 28. The liquid absorbing material 28 comprises a generally circular hollow cylinder 62 having a cavity 64 in which the non-liquid flavour generating material 30 is positioned. Thus, it will be understood that in this embodiment, the non-liquid flavour generating material 30 and the liquid absorbing material 28 are arranged separately from each other. The aerosol generating article 60 also includes a filter 66, for example comprising cellulose acetate fibres, which may help to retain the non-liquid flavour generating material 30 in the cavity 64.

In order to provide for simultaneous heating of the liquid absorbing material 28 and the non-liquid flavour generating material 30, the aerosol generating article 60 comprises an inductively heatable susceptor 32 in particulate form which is distributed both within the liquid absorbing material 28 and within the non-liquid flavour generating material 30. When a time varying electromagnetic field is produced in the vicinity of the particles of susceptor 32 by the induction coil 20, heat is generated in the susceptor 32 due to eddy currents and magnetic hysteresis losses. The heat is transferred from the particles of susceptor 32 within the liquid absorbing material 28 to the aerosol forming liquid 26 absorbed by the liquid absorbing material 28, thereby heating and atomising the absorbed aerosol forming liquid 26 to generate an aerosol 68. At the same time, the heat is transferred from the particles of susceptor 32 within the non-liquid flavour generating material 30 to the non-liquid flavour generating material 30. The non-liquid flavour generating material 30 is thereby heated without being burned and releases volatile compounds, for example including nicotine or flavour compounds 70 such as tobacco flavouring.

The aerosol 68 generated by heating the aerosol forming liquid 26 absorbed by the liquid absorbing material 28 passes out of the aerosol generating article 60. Similarly, the flavour compounds 70 generated by simultaneously heating the non-liquid flavour generating material 30 pass out of the aerosol generating article 60 through the filter 66. The aerosol 68 and flavour compounds 70 are then combined outside of the aerosol generating article 60 to form a flavoured aerosol which is inhaled by a user of the device 10, for example through a mouthpiece (not shown) fitted to the proximal end 14 of the device 10.

In the illustrated embodiment, the liquid absorbing material 28 is positioned closer to the induction coil 20 than the non-liquid flavour generating material 30 when the aerosol generating article 60 is positioned in the cavity 18. The aerosol forming liquid 26 absorbed by the liquid absorbing material 28 is thus heated to a higher temperature than the non-liquid flavour generating material 30 due to the closer proximity of the particles of susceptor 30 within the liquid absorbing material 28 to the induction coil 20.

Although exemplary embodiments have been described in the preceding paragraphs, it should be understood that various modifications may be made to those embodiments without departing from the scope of the appended claims. Thus, the breadth and scope of the claims should not be limited to the above-described exemplary embodiments.

Any combination of the above-described features in all possible variations thereof is encompassed by the present disclosure unless otherwise indicated herein or otherwise clearly contradicted by context.

Unless the context clearly requires otherwise, throughout the description and the claims, the words “comprise”, “comprising”, and the like, are to be construed in an inclusive as opposed to an exclusive or exhaustive sense; that is to say, in the sense of “including, but not limited to”.

1. An aerosol generating article comprising:
a reservoir for an aerosol forming liquid;
a liquid absorbing material for absorbing aerosol forming liquid from the reservoir; and
a non-liquid flavour generating material positioned outside the reservoir;
wherein the liquid absorbing material and the non-liquid flavour generating material are arranged to be heated simultaneously when the aerosol generating article is positioned in an aerosol generating device.

2. The aerosol generating article according to claim 1, further comprising an inductively heatable susceptor, wherein the liquid absorbing material and the non-liquid flavour generating material are arranged to be heated simultaneously by the inductively heatable susceptor when the aerosol generating article is positioned in an aerosol generating device.

3. The aerosol generating article according to claim 2, wherein the inductively heatable susceptor is distributed in the liquid absorbing material.

4. The aerosol generating article according to claim 2, wherein the inductively heatable susceptor is distributed in the non-liquid flavour generating material.

5. The aerosol generating article according to claim 1, wherein the non-liquid flavour generating material is distributed in the liquid absorbing material.

6. The aerosol generating article according to claim 1, wherein the non-liquid flavour generating material and the liquid absorbing material are arranged separately, preferably wherein the liquid absorbing material is arranged to be positioned closer to a heater or an induction coil than the non-liquid flavour generating material when the aerosol generating article is positioned in an aerosol generating device.

7. The aerosol generating article according to claim 1, wherein the non-liquid flavour generating material includes an aerosol forming liquid in an amount less than approximately 20% on a dry weight basis.

8. The aerosol generating article according to claim 1, wherein the non-liquid flavour generating material includes water in an amount less than approximately 15% on a dry weight basis.

9. The aerosol generating article according to claim 1, wherein the non-liquid flavour generating material is not positioned downstream of the liquid absorbing material relative to an aerosol flow direction within the article.

10. An aerosol generating system comprising:
an aerosol generating device comprising a cavity; and
the aerosol generating article according to claim 1 positioned in the cavity;
the aerosol generating device further comprising an atomiser for heating the aerosol generating article to generate an aerosol from aerosol forming liquid absorbed in the liquid absorbing material from the reservoir and to generate a flavour from the non-liquid flavour generating material.

11. A method for generating a flavoured aerosol, the method comprising:
providing an aerosol generating article comprising a reservoir for an aerosol forming liquid, a liquid absorbing material and a non-liquid flavour generating material positioned outside the reservoir;
transferring aerosol forming liquid from the reservoir to the liquid absorbing material;
generating an aerosol from the aerosol forming liquid absorbed in the liquid absorbing material;
generating a flavour from the non-liquid flavour generating material; and
mixing the aerosol and the flavour to generate a flavoured aerosol.

12. The method according to claim 11, wherein the step of generating an aerosol from the aerosol forming liquid absorbed in the liquid absorbing material comprises heating the liquid absorbed in the liquid absorbing material.

13. The method according to claim 11, wherein the step of generating a flavour from the non-liquid flavour generating material comprises heating the non-liquid flavour generating material.

14. The method according to claim 11, wherein the step of generating an aerosol from the aerosol forming liquid absorbed in the liquid absorbing material provides greater than approximately 85% of the total aerosol content of the flavoured aerosol.

15. The method according to claim 11, wherein the aerosol generated by the step of generating an aerosol from the aerosol forming liquid absorbed in the liquid absorbing material does not flow through the non-liquid flavour generating material.
